(19) United States
(12) Patent Application Publication
(10) Pub. No.: US 2023/0190078 A1
(43) Pub. Date: Jun. 22, 2023

(54) ASPIRATION ENDOSCOPE DEVICE AND METHODS OF USE THEREOF

(71) Applicant: THE REGENTS OF THE UNIVERSITY OF CALIFORNIA, Oakland, CA (US)

(72) Inventors: Ralph V. Clayman, Orange, CA (US); Jamie Landman, Orange, CA (US); Zachary Tano, Irvine, CA (US); Antonio R. H. Gorgen, Orange, CA (US); Andrei Cumpanas, Orange, CA (US)

(21) Appl. No.: 18/169,646

(22) Filed: Feb. 15, 2023

Related U.S. Application Data
(63) Continuation-in-part of application No. 17/738,758, filed on May 6, 2022.
Application No. 17/738,758 is a continuation-in-part of application No. 16/421,061, filed on May 23, 2019, now abandoned.
(60) Provisional application No. 63/310,281, filed on Feb. 15, 2022.
(60) Provisional application No. 62/675,929, filed on May 24, 2018.

(51) Int. Cl. A61B 1/00; A61B 1/015; A61B 1/018; A61B 1/05; A61B 1/06; A61B 10/04

(52) U.S. Cl. CPC A61B 1/00094; A61B 1/015; A61B 1/018; A61B 1/051; A61B 1/0676; A61B 1/0684; A61B 10/04

(57) ABSTRACT

The present technology relates to endoscope devices and methods of use for said endoscope devices. In one embodiment, the device comprises a semicircular aspiration channel with a blocking bar disposed at an outlet of the semicircular aspiration channel to prevent clogging of said channel. The endoscope devices of the present invention may further comprise a plurality of working channels, an image sensor, and a light source. In addition, for work within the ureter, a force sensor is incorporated to ensure safe passage of the largest flexible ureteroscope.

### CROSS-REFERENCES TO RELATED APPLICATIONS

This application is a non-provisional and claims benefit of U.S. Provisional Application No. 63/310,281 filed Feb. 15, 2022, the specification of which is/are incorporated herein in their entirety by reference.

This application is a continuation in part and claims benefit of U.S. patent application Ser. No. 17/738,758 filed May 6, 2022, which is a continuation-in-part, and claims benefit of U.S. patent application Ser. No. 16/421,061 filed May 23, 2019, which is a non-provisional and claims benefit of U.S. Provisional Application No. 62/675,929 filed May 24, 2018, the specifications of which are incorporated herein in their entirety by reference.

### FIELD OF THE INVENTION

The present invention features an endoscopic device for use in the ureter for the removal of ureteral and renal stone particle debris within a patient and methods of use for said devices which are equipped with a force sensor in order to allow for the passage of the device without injury to the ureteral wall.

### BACKGROUND OF THE INVENTION

Nephrolithiasis, or the presence of renal calculi (i.e., kidney stones) and ureteral calculi, is a common health problem across the globe, with a prevalence of 10%. The prevalence is increasing, possibly associated with the current obesity epidemic. Nephrolithiasis may lead to persistent renal obstruction, life-threatening sepsis, and permanent renal damage if left unresolved. On an annual basis, the cost to the US economy ($1.2 billion) for the care and treatment of urolithiasis patients is greater than for any other urological condition. The surgical failure to completely render the stone bearing kidney free of all stone material results in a high rate of recurrent stone disease (i.e., >40% within five years) and further patient debility and expense.

The current acute treatment options for small ureteral stones (i.e., 10 mm in diameter) nephrolithiasis include pain medication and hydration until the stone passes naturally or with pharmaceutical interventions that facilitate stone passage; approximately 70% of the smaller stones (i.e., <5 mm) will pass without the need for intervention. In instances where this approach fails or for larger ureteral stones and for all symptomatic renal stones, patients are treated with shock wave lithotripsy or ureteroscopic lithotripsy with laser probes as first-line management options, or for stones in the kidney that are larger than 2 cm, percutaneous nephrolithotomy or laparoscopic stone removal. In contrast to shock wave lithotripsy, ureteroscopy, while more invasive, is a less expensive procedure, yields higher stone-free rates, and can treat complicated, dense, or larger renal calculi up to 1.5 cm in diameter. Recent advances in ureteroscopy have also reduced complication rates (3.5%). Of note, the major drawback to both shock wave lithotripsy as well as ureteroscopy is that the stone clearance rate is no better than 60% on computed tomography (CT) scans; leaving even small stone fragments behind puts the patient at risk for recurrent stone disease. Indeed, if fragments remain, upwards of 70% of patients will require another stone removal procedure within five years, as the fragments tend to coalesce and grow. Thus, the development of a novel ureteroscope capable of thoroughly removing all fragments from the kidney would be a welcome, cost-effective advance.

In addition, the development of a larger ureteroscope, as proposed herein, with effective aspiration capabilities, would allow for the transurethral treatment of all stones in the kidney regardless of size. An advance of this nature would eliminate the need to perform percutaneous transrenal stone removal with its attendant transgression of the renal parenchyma and associated much higher (15%) and more serious complication rate (i.e., hemorrhage, transfusion, pleural injury, etc.). Also, even in the most accomplished hands, current percutaneous stone free rates for large stones are likewise disappointing (i.e., 60%). The development of the proposed ureteroscope would provide a more effective, cost-efficient, and safer approach to these larger renal stones.

Current ureteroscopes have a single common working channel that is 1.2 mm (i.e., <3.6 French) and are not equipped to provide suction evacuation of stone fragments. The small debris following laser fragmentation of a stone settles in the dependent calyces of the kidney and serves as a nidus for new stone formation, thus contributing to the recurrence of kidney stones after treatment and an ongoing cycle of treatment and recurrence. None of the currently available stone baskets can remove fragments that are <1.5 mm. At this time, reliable, complete stone clearance is only achieved with laparoscopic surgery, open surgery, or at times, with percutaneous stone removal; each of these is far more invasive than ureteroscopy and has a four-to-five-fold higher incidence of complications while also requiring an in-hospital stay (e.g., 23 hr. or longer).

Accordingly, the present invention has developed a ureteroscope with a specific central channel that is nearly three times the size of current working channels; in benchtop studies performed by the Inventors, the endoscope disclosed herein is able to evacuate all fragments that are 800 microns or smaller without clogging of the channel. Furthermore, the infection rate after ureteroscopic lithotripsy may be as high as 15%; risk factors for infection include stone size, operative duration, retained fragments that harbor bacteria, lack of use of a ureteral access sheath (e.g., lowers the infection rate to under 5%), and intrarenal pressures exceeding 40 mmHg. An endoscope capable of providing continuous flow (e.g., simultaneous inflow and aspiration) would preclude these concerning pressure spikes while eliminating the need to place a ureteral access sheath, thereby saving time and cost. Third, there are challenges with regard to the sterilization of the standard ureteroscope. Indeed, the sterilization process and repackaging of these delicate endoscopes in a hospital's surgical processing unit is injurious to the endoscope and limits its overall lifespan. There are no guidelines or quality benchmarks specific to ureteroscope reprocessing, and patient injuries and infections have been linked to the reprocessed ureteroscope itself.

### BRIEF SUMMARY OF THE INVENTION

It is an objective of the present invention to provide devices and methods that allow for an endoscope that comprises an aspiration channel (e.g., a semicircular aspiration channel) with a blocking bar disposed at an outlet of the aspiration channel to prevent clogging of the aspiration channel while ensuring the removal of all stone particle debris <1.5 mm at the end of a transurethral laser ablation of a ureteral or renal stone, as specified in the independent claims. Embodiments of the invention are given in the dependent claims. Embodiments of the present invention can be freely combined with each other if they are not mutually exclusive.

The devices and methods described herein could be used in all ureteroscopic or percutaneous nephrolithotomy laser lithotripsy procedures in which a kidney stone is broken into smaller fragments using any endoscope; however, its predominant use would be for all stones, both ureteral and renal; regardless of size. Typical endoscopes (e.g., rigid and flexible ureteroscopes or flexible nephroscopes, or flexible cystoscopes if considering bladder stones) have a small (e.g., typically 1.2 mm for ureteroscopes and 2 mm for nephroscopes and cystoscopes) central common channel that does not provide adequate suction to remove debris following stone laser fragmentation. Furthermore, all of these endoscopes have a 30° or more angulated exit from the body of the endoscope, thereby further impeding aspiration of any fragments; indeed, the channel as it exits the body of the endoscope is further reduced in size in order to accept a female Luer-lok adaptor. The endoscope device of the present invention comprises an aspiration channel that comprises nearly 80% of the circumference of the endoscope. For example, a 13.5 Fr endoscope would have a semicircular 9 Fr (i.e., 3 mm in diameter) aspiration channel with aspiration capabilities for fragments <1.5 mm even with the central blocking bar. The semicircular design has been documented in laboratory work by the Inventors as having the best configuration for the aspiration of stone fragments. The endoscope device herein may hold the CCD or CMOS visualizing chip and would also be illuminated using one or two LED lights. The endoscope device may comprise a plurality of working channels (e.g., two working channels, e.g., with one dedicated to the passage of a laser fiber and the other for irrigation and/or passage of a stone basket or guidewire); as such, the surgeon will be able to provide for continuous flow (e.g., active irrigation and passive/active aspiration) simultaneous with the laser fragmentation/dusting of the stone. This will allow for ongoing aspiration of small fragments created by the lasering of the stone and will also keep the pressure in the renal pelvis well below 40 mmHg, which is the threshold for pyelovenous/pyelosinus/pyelolymphatic backflow and possible sepsis. The handle of the endoscope will have control buttons for aspiration (activation/deactivation) and for irrigation (activation/deactivation), as well as an entry point for the laser fiber, which can also be advanced or retracted with a control button. The aspiration channel would exit the back end of the endoscope, and the suction tubing would go over the point of exit, thereby not narrowing the channel at all while providing the most direct path of exit for the aspirated stone dust/fragments. The endoscope device would also have a self-contained battery pack and is Bluetooth™/Wi-Fi enabled, thereby eliminating all electrical cords and cables and providing for image transmissions to all devices (e.g., television screen, digital pad, or phone). The irrigation tubing comes off of the endoscope at 6 o'clock and directly posterior (e.g., 90 degrees to the handle), away from the surgeon's hands, thereby adding to the ease of use and balance of the endoscope.

In some embodiments, the present invention features an endoscope device comprising an insertion tube comprising an insertion tip at a distal end of the insertion tube, an aspiration channel disposed in the insertion tube and having an outlet at the insertion tip, at least one blocking bar disposed at the outlet of the aspiration channel, a plurality of working channels disposed in the insertion tube each having an outlet at the insertion tip, an image sensor and a light source disposed at the insertion tip. In some embodiments, the at least one blocking bar splits the outlet of the aspiration channel into multiple openings. In addition, in some embodiments, the present invention, either in a rigid or flexible rendition, has a small battery and transmitter housed within a portion of the insertion tip opposite of the aspiration channel of the endoscope device rather than in the handle, such that the entire instrument is self-contained and disposable.

One of the unique and inventive technical features of the present invention is a blocking bar disposed across (e.g., centrally or angulated) the aspiration channel to preclude clogging of the aspiration channel by stone fragments. Without wishing to limit the invention to any theory or mechanism, it is believed that the technical feature of the present invention advantageously provides for the prevention of clogging of the aspiration channel. Additionally, because the blocking bar divides the large (e.g., semicircular) aspiration channel, the endoscope device can be advanced over two guidewires, thereby facilitating the passage of the endoscope. Furthermore, none of the prior arts teaches of a blocking bar dividing a large aspiration channel. None of the presently known prior references or work has the unique inventive technical feature of the present invention.

Furthermore, the prior references teach away from the present invention. For example, prior references do not teach the use of endoscopes (e.g., ureteroscopes) larger than 10 Fr. From the extensive research by the Inventors regarding ureteral distensibility, unique clinical knowledge has been obtained which reveals that nearly 90% of ureters would accept an 11.5 Fr endoscope, while 80% would accept a 13.5 Fr ureteroscope. Indeed, 58% of ureters would accept a 15.5 Fr ureteroscope. Additionally, prior references do not teach endoscopes (e.g., ureteroscopes) that are 45 cm long for use, specifically in female patients.

The combination of creating disposable ureteroscopes of the aforedescribed design in both 45 cm and 65 cm lengths and 3 or 4 different diameters (e.g., 9.5 Fr, 11.5 Fr, 13.5 Fr, and 15.5 Fr) allows the urologist for the first time to select an endoscope to use based on the patient's gender and the distensibility of the ureter as measured at the time of surgery, thereby affording the safe passage of a larger, more efficient, disposable ureteroscope.

Furthermore, the prior references teach away from the present invention. For example, prior references do not teach of the triple lumen device comprising a large (e.g., semicircular) aspiration channel and two smaller working channels, e.g., a dedicated laser port and a port for passage of a biopsy forceps or stone basket, as well as for ongoing irrigation.

Furthermore, the inventive technical features of the present invention contributed to a surprising result, in that the presence of the multiple channels enabled the development of parameters such that a continuous flow situation could be invoked whereby the level of aspiration could be balanced with the flow of irrigant to allow ongoing suction of fragments produced by the laser while simultaneously maintaining a clear field of view which will markedly expedite the stone removal procedure while also improving its effectiveness.

Additionally, as described herein, the flexible disposable ureteroscope comes equipped with a force sensor on the working channel through which a guidewire is passed. As such, as the endoscope is advanced up the ureter, the urologist is informed when 3 Newtons (no ureteral trauma or edema should occur—hence no need to place an indwelling stent), 6 Newtons (no risk of splitting the urothelium but will need to leave an indwelling ureteral stent, should stop at this point and downsize to a smaller ureteroscope), and 8 Newtons (real risk of splitting the urothelium, stop and downsize to a smaller ureteroscope, and will need to leave a ureteral stent).

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, and 1C show the insertion tip (i.e., the distal end) of an endoscope device comprising two working channels (e.g., one working channel is dedicated to the passage of a laser, and the other (e.g., the larger port) is dedicated to irrigation). The endoscope device further comprises an aspiration channel (e.g., a semicircular aspiration channel) with a blocking bar disposed at the outlet of the aspiration channel. The blocking bar splits the aspiration channel into two equal openings. The working channels as well as the aspiration channel extend from the distal end of the endoscope device to the proximal end of the endoscope device. FIG. 1A shows the distal end of the endoscope device in which the blocking bar splits the outlet of the aspiration channel equally and is centrally disposed at the outlet of the aspiration channel at a 90° angle. FIGS. 1B and 1C show the endoscope device described herein with (FIG. 1B) or without (FIG. 1C) a divider disposed longitudinally within the insertion tube such that an interior space of the insertion tube is divided into two lumens, wherein one of the lumens forms the aspiration channel and the other lumen contains the working channels, the image sensor, and the light source.

FIGS. 2A, 2B, and 2C show the insertion tip (i.e., the distal end) of an endoscope device comprising two working channels (e.g., one working channel is dedicated to the passage of a laser, and the other (e.g., the larger port) is dedicated to irrigation). The endoscope device further comprises an aspiration channel (e.g., a semicircular aspiration channel) with a blocking bar disposed at the outlet of the aspiration channel. The blocking bar splits the aspiration channel into two openings. The working channels as well as the aspiration channel extend from the distal end of the endoscope device to the proximal end of the endoscope device. FIG. 2A shows the distal end of the endoscope device in which the blocking bar splits the outlet of the aspiration channel unequally and is disposed at the outlet of the aspiration channel at a 60° angle. FIGS. 2B and 2C show the endoscope device described herein with (FIG. 2B) or without (FIG. 2C) a divider disposed longitudinally within the insertion tube such that an interior space of the insertion tube is divided into two lumens, wherein one of the lumens forms the aspiration channel and the other lumen contains the working channels, the image sensor, and the light source.

FIGS. 3A, 3B, and 3C show the insertion tip (i.e., the distal end) of an endoscope device comprising two working channels (e.g., one working channel is dedicated to the passage of a laser, and the other (e.g., the larger port) is dedicated to irrigation). The endoscope device further comprises an aspiration channel (e.g., a semicircular aspiration channel) with a blocking bar disposed at the outlet of the aspiration channel. The blocking bar splits the aspiration channel into two openings. The working channels as well as the aspiration channel extend from the distal end of the endoscope device to the proximal end of the endoscope device. FIG. 3A shows the distal end of the endoscope device in which the blocking bar splits the outlet of the aspiration channel unequally and is disposed at the outlet of the aspiration channel at a 30° angle. FIGS. 3B and 3C show the endoscope device described herein with (FIG. 3B) or without (FIG. 3C) a divider disposed longitudinally within the insertion tube such that an interior space of the insertion tube is divided into two lumens, wherein one of the lumens forms the aspiration channel and the other lumen contains the working channels, the image sensor, and the light source.

### DETAILED DESCRIPTION OF THE INVENTION

Following is a list of elements corresponding to a particular element referred to herein:
100 Endoscope Device
101 Distal End
102 Proximal End
110 Aspiration Channel
115 Aspiration Channel Outlet
120 Blocking Bar
130 Image Sensor
140 Light Source
150 Working Channel
155 Working Channel Outlet
160 Insertion Tube
165 Insertion Tip
170 Divider

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

A “subject” is an individual and includes, but is not limited to, a mammal (e.g., a human, horse, pig, rabbit, dog, sheep, goat, non-human primate, cow, cat, guinea pig, or rodent), a fish, a bird, a reptile, or an amphibian. The term does not denote a particular age or sex. A “patient” is a subject afflicted with a disease or disorder. The term “patient” includes human and veterinary subjects.

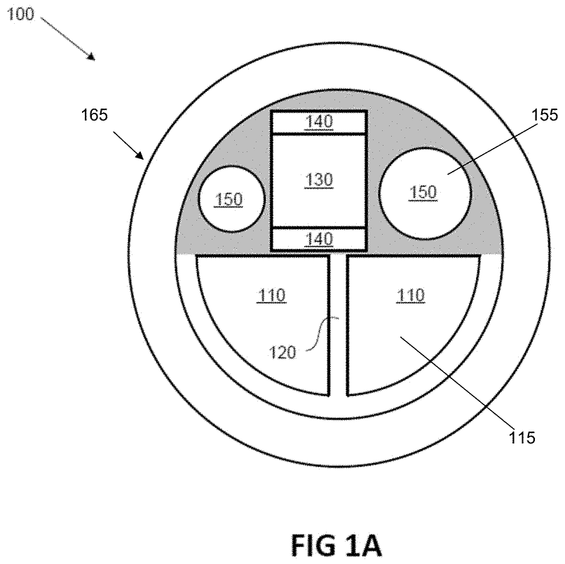

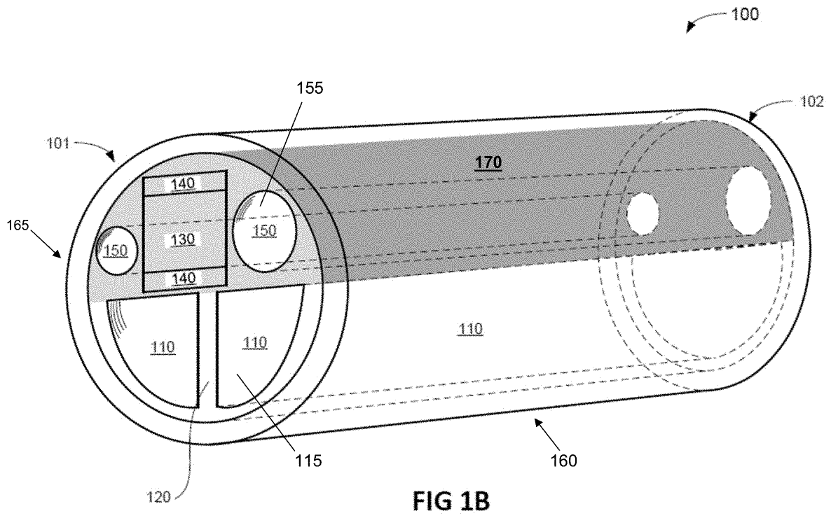

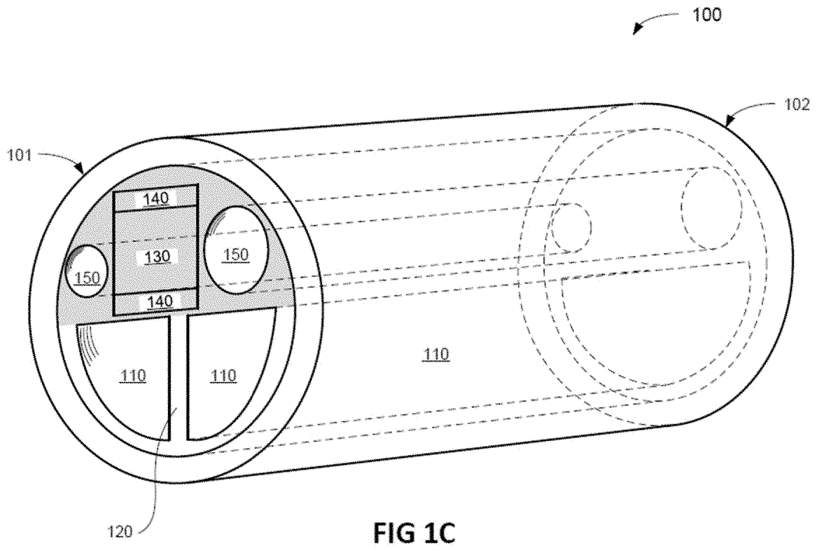

Referring now to FIGS. 1A, 1B, 10, 2A, 2B, 2C, 3A, 3B, and 3C, the present invention features devices comprising a unique, large aspiration channel (e.g., a semicircular aspiration channel) for the removal of all stone particle debris within a patient during ureteroscopic laser lithotripsy and methods of use for said devices.

The present invention features an endoscope device (100) comprising an insertion tube (160) comprising an insertion tip (165) at a distal end (101) of the insertion tube (160), an aspiration channel (110) disposed in the insertion tube (160) and having an outlet (115) at the insertion tip (165), at least one blocking bar (120) disposed at the outlet (115) of the aspiration channel (110), a plurality of working channels (150) disposed in the insertion tube (160) each having an outlet (155) at the insertion tip (165), an image sensor (130) and a light source (140) disposed at the insertion tip (165). In some embodiments, the at least one blocking bar (120) splits the outlet (115) of the aspiration channel (110) into multiple openings.

In some embodiments, the endoscope device (100) comprises a rigid endoscope. In other embodiments, the endoscope device (100) comprises a flexible endoscope. In some embodiments, the endoscope device (100) is a ureteroscope. In some embodiments, the endoscope device (100) is a nephroscope. In some embodiments, the endoscope device (100) is rigid or flexible.

In some embodiments, the endoscope device (100) is 45 cm in length, i.e., is 45 cm in length from the distal end (101) of the endoscope device (100) to the proximal end (102) of the endoscope device (100). In some embodiments, the endoscope device (100) is 65 cm in length, i.e., is 65 cm in length from the distal end (101) of the endoscope device (100) to the proximal end (102) of the endoscope device (100). In some embodiments, the endoscope device (100) is about 25 cm to 75 cm, or about 25 cm to 65 cm, or about 25 cm to 55 cm, or about 25 cm to 45 cm, or about 25 cm to 35 cm, or about 35 cm to 75 cm, or about 35 cm to 65 cm, or about 35 cm to 55 cm, or about 35 cm to 45 cm, or about 45 cm to 75 cm, or about 45 cm to 65 cm, or about 45 cm to 55 cm, or about 55 cm to 75 cm, or about 55 cm to 65 cm, or about 65 cm to 75 cm in length.

In certain embodiments, the endoscope device (100) comprises an insertion tube (160) having a diameter of 8 Fr to 20 Fr. In other embodiments, the endoscope device (100) comprises an insertion tube (160) having a diameter of about 8 Fr to 20 Fr, or about 8 Fr to 19 Fr, or about 8 Fr to 18 Fr, or about 8 Fr to 17 Fr, or about 8 Fr to 16 Fr, or about 8 Fr to 15 Fr, or about 8 Fr to 14 Fr, or about 8 Fr to 13 Fr, or about 8 Fr to 12 Fr, or about 8 Fr to 11 Fr, or about 8 Fr to about 10 Fr, or about 8 Fr to 9 Fr. In some embodiments, the endoscope device (100) comprises an insertion tube (160) having a diameter of about 9 Fr to 20 Fr, or about 9 Fr to 19 Fr, or about 9 Fr to 18 Fr, or about 9 Fr to 17 Fr, or about 9 Fr to 16 Fr, or about 9 Fr to 15 Fr, or about 9 Fr to 14 Fr, or about 9 Fr to 13 Fr, or about 9 Fr to 12 Fr, or about 9 Fr to 11 Fr, or about 9 Fr to about 10 Fr. In some embodiments, the endoscope device (100) comprises an insertion tube (160) having a diameter of about 10 Fr to 20 Fr, or about 10 Fr to 19 Fr, or about 10 Fr to 18 Fr, or about 10 Fr to 17 Fr, or about 10 Fr to 16 Fr, or about 10 Fr to 15 Fr, or about 10 Fr to 14 Fr, or about 10 Fr to 13 Fr, or about 10 Fr to 12 Fr, or about 10 Fr to 11 Fr. In some embodiments, the endoscope device (100) comprises an insertion tube having a diameter of about 11 Fr to 20 Fr, or about 11 Fr to 19 Fr, or about 11 Fr to 18 Fr, or about 11 Fr to 17 Fr, or about 11 Fr to 16 Fr, or about 11 Fr to 15 Fr, or about 11 Fr to 14 Fr, or about 11 Fr to 13 Fr, or about 11 Fr to 12 Fr. In some embodiments, the endoscope device (100) comprises an insertion tube (160) having a diameter of about 12 Fr to 20 Fr, or about 12 Fr to 19 Fr, or about 12 Fr to 18 Fr, or about 12 Fr to 17 Fr, or about 12 Fr to 16 Fr, or about 12 Fr to 15 Fr, or about 12 Fr to 14 Fr, or about 12 Fr to 13 Fr. In some embodiments, the endoscope device (100) comprises an insertion tube (160) having a diameter of about 13 Fr to 20 Fr, or about 13 Fr to 19 Fr, or about 13 Fr to 18 Fr, or about 13 Fr to 17 Fr, or about 13 Fr to 16 Fr, or about 13 Fr to 15 Fr, or about 13 Fr to 14 Fr, or about 14 Fr to 20 Fr, or about 14 Fr to 19 Fr, or about 14 Fr to 18 Fr, or about 14 Fr to 17 Fr, or about 14 Fr to 16 Fr, or about 14 Fr to 15 Fr. In some embodiments, the endoscope device (100) comprises an insertion tube (160) having a diameter of about 15 Fr to 20 Fr, or about 15 Fr to 19 Fr, or about 15 Fr to 18 Fr, or about 15 Fr to 17 Fr, or about 15 Fr to 16 Fr, or about 16 Fr to 20 Fr, or about 16 Fr to 19 Fr, or about 16 Fr to 18 Fr, or about 16 Fr to 17 Fr, or about 17 Fr to 20 Fr, or about 17 Fr to 19 Fr, or about 17 Fr to 18 Fr, or about 18 Fr to 20 Fr, or about 18 Fr to 19 Fr, or about 19 Fr to 20 Fr. In further embodiments, the insertion tubes (110) described herein are 8 Fr, or 9 Fr, or 10 Fr, or 11 Fr, or 12 Fr, or 13 Fr, or 14 Fr, or 15 Fr, or 16 Fr, or 17 Fr, or 18 Fr, or 19 Fr, or 20 Fr. In some embodiments, the insertion tubes (110) described herein are larger than 20 Fr.

In some embodiments, the outlet (115) of the aspiration channel (110) occupies at least half of a space on the insertion tip (165), and the working channels (150), image sensor (130), and light source (140) occupy the remaining space on the insertion tip (165). In some embodiments, the outlet (115) of the aspiration channel (110) occupies half of the space on the insertion tip (165), and the working channels (150), image sensor (130), and light source (140) occupy the remaining space on the insertion tip. In some embodiments, the outlet (115) of the aspiration channel (110) occupies more than half of the space on the insertion tip (165), and the working channels (150), image sensor (130), and light source (140) occupy the remaining space on the insertion tip (165).

In some embodiments, the aspiration channel (110) is a large bore channel. In some embodiments, the endoscope devices (100) described herein feature at least one large bore (e.g., 7 Fr to 15 Fr) aspiration channel for optimal aspiration, such as to evacuate all stone fragments <1.5 mm or larger remnants from laser lithotripsy during ureteroscopy.

In some embodiments, the outlet (115) of the aspiration channel (110) is semicircular in shape. In some embodiments, the outlet (115) of the aspiration channel (110) may further comprise a filter (e.g., a sieve-like filter; e.g., a sieve-like semicircular filter) disposed at the insertion tip (165). In some embodiments, the aspiration channel (110) is semicircular in shape. In other embodiments, the aspiration channel (110) is irregular in shape.

In some embodiments, the aspiration channel (110) is 7 Fr to 15 Fr. In other embodiments, the aspiration channel (110) is about 6 Fr to 16 Fr, or about 6 Fr to 15 Fr, or about 6 Fr to 14 Fr, or about 6 Fr, to 13 Fr, or about 6 Fr to 12 Fr, or about 6 Fr to 11 Fr, or about 6 Fr to 10 Fr, or about 6 Fr to 9 Fr, or about 6 Fr to 8 Fr, or about 6 Fr to 7 Fr, or about 7 Fr to 16 Fr, or about 7 Fr to 15 Fr, or about 7 Fr to 14 Fr, or about 7 Fr, to 13 Fr, or about 7 Fr to 12 Fr, or about 7 Fr to 11 Fr, or about 7 Fr to 10 Fr, or about 7 Fr to 9 Fr, or about 7 Fr to 8 Fr, or about 8 Fr to 16 Fr, or about 8 Fr to 15 Fr, or about 8 Fr to 14 Fr, or about 8 Fr, to 13 Fr, or about 8 Fr to 12 Fr, or about 8 Fr to 11 Fr, or about 8 Fr to 10 Fr, or about 8 Fr to 9 Fr, or about 9 Fr to 16 Fr, or about 9 Fr to 15 Fr, or about 9 Fr to 14 Fr, or about 9 Fr, to 13 Fr, or about 9 Fr to 12 Fr, or about 9 Fr to 11 Fr, or about 9 Fr to 10 Fr, or about 10 Fr to 16 Fr, or about 10 Fr to 15 Fr, or about 10 Fr to 14 Fr, or about 10 Fr, to 13 Fr, or about 10 Fr to 12 Fr, or about 10 Fr to 11 Fr, or about 11 Fr to 16 Fr, or about 11 Fr to 15 Fr, or about 11 Fr to 14 Fr, or about 11 Fr, to 13 Fr, or about 11 Fr to 12 Fr, or about 12 Fr to 16 Fr, or about 12 Fr to 15 Fr, or about 12 Fr to 14 Fr, or about 12 Fr, to 13 Fr, or about 13 Fr to 16 Fr, or about 13 Fr to 15 Fr, or about 13 Fr to 14 Fr, or about 14 Fr to 16 Fr, or about 14 Fr to 15 Fr, or about 15 Fr to 16 Fr. In some embodiments, the aspiration channels (110) described herein are smaller than 6 Fr. In other embodiments, the aspiration channels (110) described herein are larger than 15 Fr.

In some embodiments, the aspiration channel (110) extends linearly from the distal end (101) to the proximal end (102) of the endoscope device (e.g., the ureteroscope) such that no sharp angles are formed within the aspiration channel (110). In some embodiments, the aspiration channel (110) has a constant diameter throughout the length of the endoscope device (100; e.g., a constant diameter from the distal end (101) to the proximal end (102)). Additionally, there may be limited narrowing (or no narrowing) of the aspiration channel (110) at the proximal end (102) where the suction tubing is connected to the endoscope device (100; e.g., a ureteroscope)—as such, the exit connection would be as a male connector over which the suction tubing would be placed.

In embodiments in which a flexible endoscope is used, the aspiration channel (110) is designed in a fashion to prevent narrowing when the flexible endoscope (e.g., ureteroscope) is flexed to prevent the impedance of stone passage.

In some embodiments, the endoscope devices (100) described herein comprise an aspiration channel (110) adapted to capture a stone (e.g., a ureteral or kidney stone) of 0.25 mm to 1.5 mm. In other embodiments, the endoscope devices (100) described herein comprise an aspiration channel (110) adapted to capture a stone (e.g., a kidney stone) of about 0.10 mm to 2.0 mm, or about 0.10 mm to 1.8 mm, or about 0.10 mm to 1.6 mm, or about 0.10 mm to 1.5 mm, or about 0.10 mm to 1.4 mm, or about 0.10 mm to 1.2 mm, or about 0.10 mm to 1.0 mm, or about 0.1 mm to 0.5 mm, or about 0.10 mm to 0.25 mm, or about 0.10 to 0.20 mm, or about 0.20 mm to 2.0 mm, or about 0.20 to 1.8 mm, or about 0.20 mm to 1.6 mm, or about 0.20 mm to 1.5 mm, or about 0.20 mm to 1.4 mm, or about 0.20 mm to 1.2 mm, or about 0.20 mm to 1.0 mm, or about 0.20 mm to 0.5 mm, or about 0.20 mm to 0.25 mm, or about 0.25 mm to 2.0 mm, or about 0.25 mm to 1.8 mm, or about 0.25 mm to 1.6 mm, or about 0.25 mm to 1.5 mm, or about 0.25 mm to 1.4 mm, or about 0.25 mm to 1.2 mm, or about 0.25 mm to 1.0 mm, or about 0.25 mm to 0.5 mm, or about 0.5 mm to 2.0 mm, or about 0.5 to 1.8 mm, or about 0.5 mm to 1.6 mm, or about 0.5 mm to 1.5 mm, or about 0.5 mm to 1.4 mm, or about 0.5 mm to 1.2 mm, or about 0.5 mm to 1.0 mm, or about 1.0 mm to 2.0 mm, or about 1.0 mm to 1.8 mm or about 1.0 mm to 1.6 mm, or about 1.0 mm to 1.5 mm, or about 1.0 mm to 1.4 mm, or about 1.0 mm to 1.2 mm, or about 1.5 mm to 2.0 mm, or about 1.5 mm to 1.8 mm, or about 1.5 mm to 1.6 mm, or about 1.4 mm to 2.0 mm, or about 1.4 mm to 1.8 mm or about 1.4 mm to 1.6 mm, or about 1.6 mm to 2.0 or about 1.6 mm to 1.8 mm, or about 1.8 mm to 2.0 mm.

In further embodiments, the endoscope devices (100) described herein comprise an aspiration channel (110) adapted to capture a stone (e.g., a kidney stone) that is 0.1 mm, 0.2 mm, 0.25 mm, or 0.4 mm, or 0.5, mm, or 0.6 mm, or 0.7 mm, or 0.8 mm, or 0.9 mm, or 1.0 mm, or 1.1 mm, or 1.2 mm, or 1.3 mm, or 1.4 mm, or 1.5 mm, or 1.6 mm, or 1.7 mm, or 1.8 mm, or 1.9 mm, or 2.0 mm. In some embodiments, the devices (100) described herein comprise an aspiration channel (110) adapted to capture a stone (e.g., a kidney stone) that is smaller than 0.1 mm. In other embodiments, the devices (110) described herein comprise an aspiration channel (110) adapted to capture a stone (e.g., a kidney stone) that is larger than 2.0 mm. In some embodiments, stones larger than 2.0 mm are entrapped in a stone basket and removed intact, e.g., from a working channel (150). In some embodiments, stones larger than 1.5 mm are entrapped in a stone basket and removed intact, e.g., from a working channel (150).

In some embodiments, the endoscope devices (100) described herein comprise an aspiration channel (110) that is adapted for a suction form of biopsy of a polyp and/or tumor in a subject. In other embodiments, the endoscope devices (100) described herein comprise an aspiration channel (110) adapted for suction in a bronchus, colon, or any area of the gastrointestinal tract of a subject or introduced to be used as an angioscope, biliary endoscope, laryngoscope or in a smaller version for the salivary ducts, spinal canal or for arthroscopy or during laparoscopic/robotic surgery.

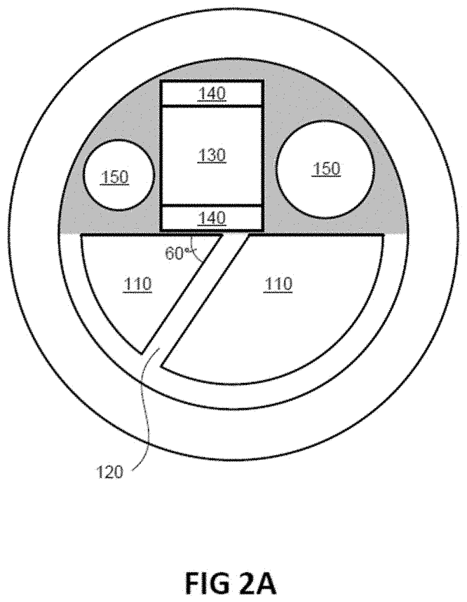

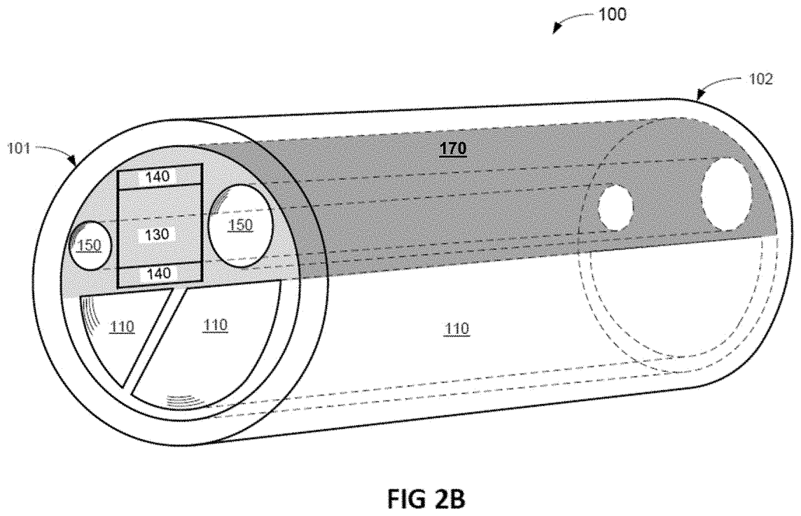

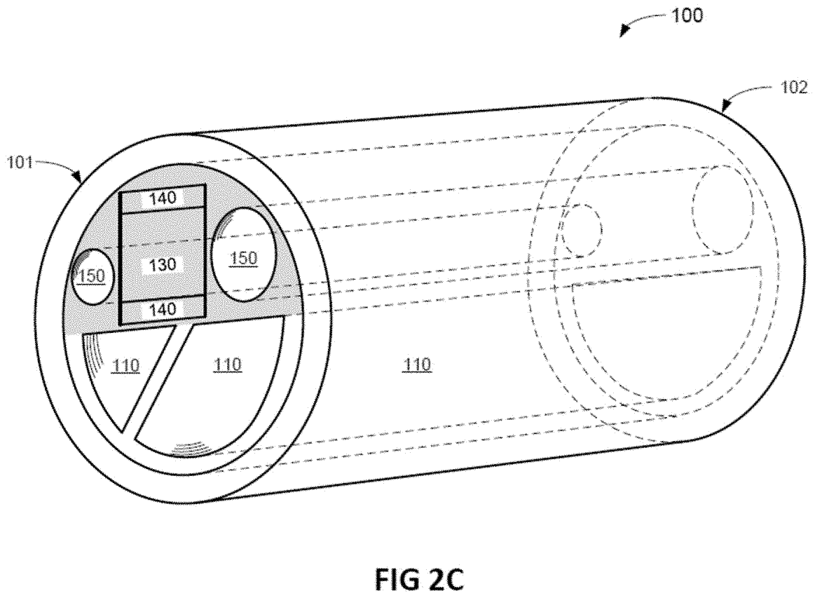

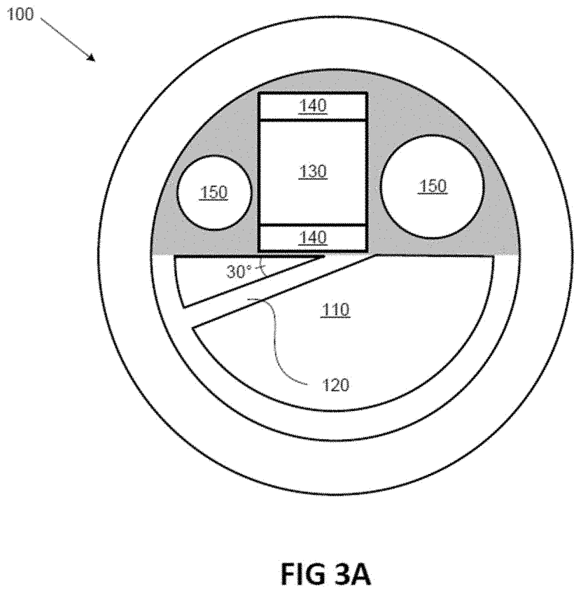

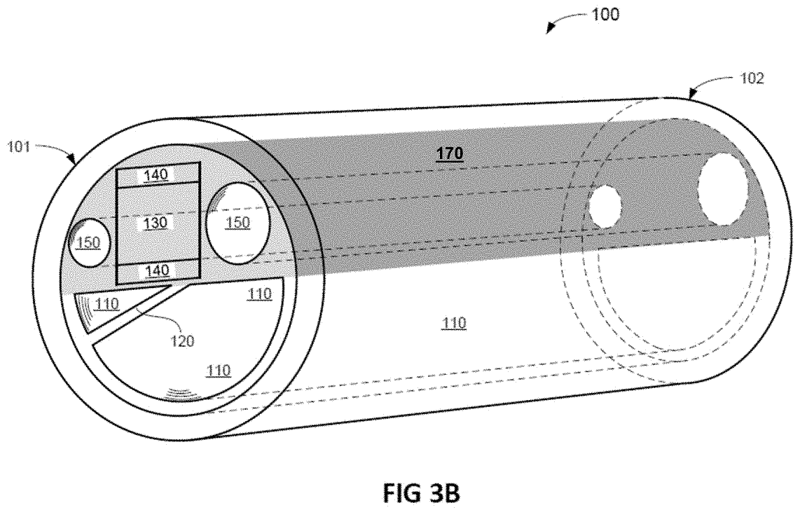

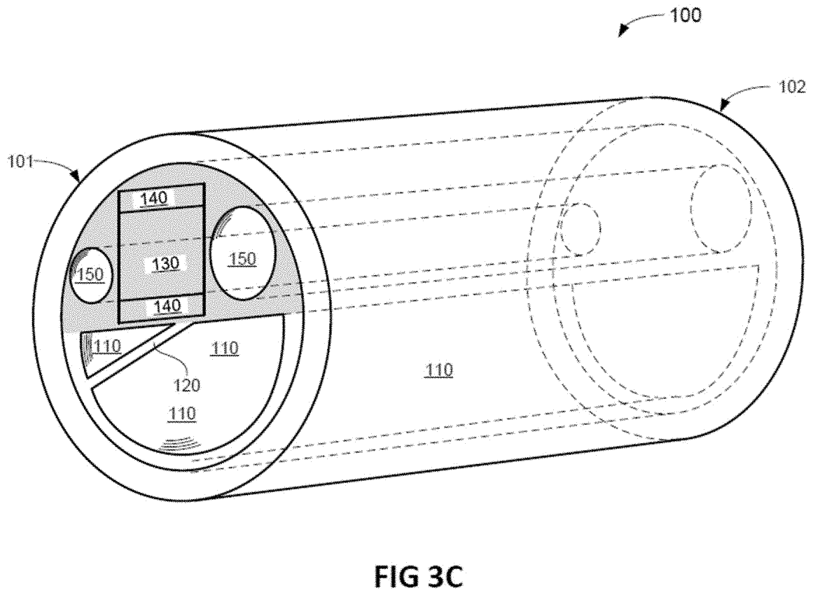

In some embodiments, the at least one blocking bar (120) splits the outlet (115) of the aspiration channel (110) equally. In some embodiments, the at least one blocking bar (120) splits the outlet (115) of the aspiration channel (110) unequally. Accordingly, the device may comprise two blocking bars, or the lone blocking bar may be placed at a 30° or 60° angle, allowing for slightly larger fragments to be aspirated. In some embodiments, the at least one blocking bar (120) is centrally disposed at the outlet (115) of the aspiration channel (110) at a 90° angle (FIGS. 1A, 1B, and 10). In some embodiments, the at least one blocking bar (120) disposed at the outlet (115) of the aspiration channel (110) at a 60° angle (FIGS. 2A, 2B, and 2C) or 30° angle (FIG. FIGS. 3A, 3B, and 3C). In some embodiments, the endoscope device (100) comprises one blocking bar (120). In some embodiments, the endoscope device (100) comprises two blocking bars (120). In some embodiments, the endoscope device (100) comprises three blocking bars (120). In some embodiments, the endoscope device (100) comprises four blocking bars (120). Without wishing to limit the present invention to any theory or mechanism, it is believed that the blocking bar (120), as described herein, prevents clogging of the aspiration channel (110).

In some embodiments, the endoscope devices (100) described herein comprise two channels (e.g., a singular aspiration channel (110) and a singular working channel (150)). In some embodiments, the endoscope devices (100) described herein comprise three channels (e.g., a singular aspiration channel (110) and two working channels (150)). In other embodiments, endoscope devices (100) described herein comprise four channels, e.g., two aspiration channels (110) and two working channels (150), or alternatively one aspiration channel (110) and three working channels (150).

In some embodiments, the plurality of working channels (150) extends from the distal end (101) of the endoscope device (100) to a proximal end (102) of the endoscope device (100). In some embodiments, the plurality of working channels (150) may exit the handle of the endoscope device at differing angles. In one embodiment, in which the endoscope device (100) comprises two working channels (e.g., a laser port and a separate irrigation port), the two working channels (150) may extend from the distal end (101) to the proximal end (102) and exit the handle of the endoscope device (100) at a 60° or 90° angle. For example, a working channel (150) comprising a laser port may exit the handle at a 60° angle, whereas a working channel (150) comprising an irrigation port may exit the handle at a 90° angle.

In some embodiments, the handle of the endoscope device (100) may comprise three control buttons: (1) to advance/retract the laser fiber, (2) to initiate or stop irrigant flow, and (3) to initiate or stop aspiration.

In some embodiments, the plurality of working channels (150) comprises an irrigation port, a stone basket port, a biopsy port, a laser port, or a combination thereof. In other embodiments, the plurality of working channels (150) comprises an irrigation port and an equipment port. Non-limiting examples of equipment may include but are not limited to a stone basket, guide wire, laser fiber, biopsy forceps, brush, a two- or three-pronged grasper, or a combination thereof.

In certain embodiments, the working channels (150) of the endoscope device (100) described herein have an inner diameter (ID) of 0.6 mm to 0.8 mm. In some embodiments, the working channels (150) of the device (100) described herein have an inner diameter (ID) of about 0.5 mm to 1.2 mm, or about 0.5 mm to 1.1 mm, or about 0.5 mm to 1.0 mm, or about 0.5 mm to 0.9 mm, or about 0.5 mm to 0.8 mm, or about 0.5 mm to 0.7 mm, or about 0.5 mm to 0.6 mm, or about 0.6 mm to 1.2 mm, or about 0.6 mm to 1.1 mm, or about 0.6 mm to 1.0 mm, or about 0.6 mm to 0.9 mm, or about 0.6 mm to 0.8 mm, or about 0.6 mm to 0.7 mm, or about 0.7 mm to 1.2 mm, or about 0.7 mm to 1.1 mm, or about 0.7 mm to 1.0 mm, or about 0.7 mm to 0.9 mm, or about 0.7 mm to 0.8 mm, or about 0.8 mm to 1.2 mm, or about 0.8 mm to 1.1 mm, or about 0.8 mm to 1.0 mm, or about 0.8 mm to 0.9 mm, or about 0.9 mm to 1.2 mm, or about 0.9 mm to 1.1 mm, or about 0.9 mm to 1.0 mm, or about 1.0 mm to 1.2 mm, or about 1.0 mm to 1.1 mm, or about 1.1 mm to 1.2 mm.

In some embodiments, the insertion tube (160) comprises a plurality of working channels (150). In some embodiments, the insertion tube (160) of the endoscope device (100) comprises two working channels (150). In some embodiments, the insertion tube (160) of the endoscope device (100) comprises one working channel (150). In other embodiments, the insertion tube (160) of the endoscope device (100) comprises three working channels (150). In some embodiments, the insertion tube (160) of the endoscope device (100) comprises four working channels (150).

In some embodiments, the outlets (155) of the working channels (150) are flush with the insertion tip (165; e.g., at the distal end (101)) of the endoscope device (100). In other embodiments, the outlets (155) of the working channels (150) extend past the insertion tip (165; e.g., the distal end (101)) of the endoscope device (100) to preclude aspiration of the urothelium and potential plugging of the aspiration channel (110).

In certain embodiments, a portion of the insertion tip (165) at the distal end (101) of the insertion tube (160) opposite of the aspiration channel (110) is recessed into the insertion tip (165; e.g., the distal end (101)) of the endoscope device (100)). In some embodiments, the portion of the insertion tip (165) at the distal end (101) of the insertion tube (160) opposite of the aspiration channel (110) is recessed into the insertion tip (165) up to 1 cm. In other embodiments, the portion of the insertion tip (165) at the distal end (101) of the insertion tube (160) opposite of the aspiration channel (110) is recessed into the insertion tip (165) about 0.1 cm, or about 0.2 cm, or about 0.3 cm, or about 0.4 cm, or about 0.5 cm, or about 0.6 cm, or about 0.7 cm, or about 0.8 cm, or about 0.9 cm, or about 1.0 cm. In other embodiments, a portion of the insertion tip (165) at the distal end (101) of the insertion tube (160) opposite of the aspiration channel (110) extends past the insertion tip (165) (e.g., the distal end (101)) of the endoscope device (100). In some embodiments, a portion of the insertion tip (165) at the distal end (101) of the insertion tube (160) opposite of the aspiration channel (110) extends past the insertion tip (165) up to 1 cm. In other embodiments, a portion of the insertion tip (165) at the distal end (101) of the insertion tube (160) opposite of the aspiration channel (110) extends past the insertion tip (165) by about 0.1 cm, or about 0.2 cm, or about 0.3 cm, or about 0.4 cm, or about 0.5 cm, or about 0.6 cm, or about 0.7 cm, or about 0.8 cm, or about 0.9 cm, or about 1.0 cm.

Without wishing to limit the present invention to any theories or mechanisms, it is believed that when a portion of the insertion tip (165) at the distal end extends past the insertion tip (165; e.g., the distal end (101) of the endoscope device (100)), enables that portion of the insertion tip (165) to act as a “brush” to brush off fragments from the urothelium and then suction them into the endoscope. In other embodiments, the devices (100) described herein comprise bristles along the aspiration channel (110) to brush off fragments from the urothelium and then suction them into the endoscope. In some embodiments, the bristles along the aspiration channel (110) would not impede the passage of the endoscope device (100). In some embodiments, the devices described herein comprising bristles along the aspiration channel (110) further comprise a blocking bar; (120)) that is flush with the distal end (101) of the device (100). In further embodiments, the working channels (150) of the endoscope device (100) comprise bristles on the outer surface of the working channels (150) to facilitate the release and subsequent aspiration of fragments from the urothelium.

In some embodiments, the insertion tube (160) further comprises a divider (170) disposed longitudinally (e.g., along the length of the endoscope device (100); i.e., from the distal end (101) to the proximal end (102)) within the insertion tube (160) such that the interior space of the insertion tube (160) is divided into two lumens. One of the lumens forms the aspiration channel (110), and the other lumen contains the plurality of working channels (150), the image sensor (130), and the light source (140).

In some embodiments, the image sensor (130) is battery operated. In some embodiments, the image sensor (130) is wireless. In some embodiments, the light source (140) is battery operated. In some embodiments, the light source (140) is wireless. In some embodiments, wireless video capabilities via Bluetooth™ and/or WiFi, thereby eliminating entangling cords and expensive high-power light sources and fixed camera equipment (e.g., an image sensor).

In some embodiments, the endoscope device (100) comprises one light source (140). In other embodiments, the endoscope device (100) comprises two light sources (140). In further embodiments, a portion of the insertion tip (165) at the distal end (101) of the insertion tube (160) opposite the aspiration channel (110) is made of material that illuminates. In other embodiments, a portion of the insertion tip (165) at the distal end (101) of the insertion tube (160) opposite of the aspiration channel (110) is made of material that absorbs or reflects light from a laser and then gives off said light. In some embodiments, the light source (140) comprises an LED (light-emitting diode). In other embodiments, the light source (140) comprises a fiber optic.

In certain embodiments, the light source (140) is 0.65 mm square (sq). In other embodiments, the light source (140) is about 0.20 mm sq to 1.1 mm sq, or about 0.20 mm sq to 0.95 mm sq, or about 0.20 mm sq to 0.80 mm sq, or about 0.20 mm sq to 6.5 mm sq, or about 0.20 mm sq to 5.0 mm sq, or about 0.20 mm sq to 0.35 mm sq, or about 0.35 mm sq to 1.1 mm sq, or about 0.35 mm sq to 0.95 mm sq, or about 0.35 mm sq to 0.80 mm sq, or about 0.35 mm sq to 6.5 mm sq, or about 0.35 mm sq to 5.0 mm sq, or about 5.0 mm sq to 1.1 mm sq, or about 5.0 mm sq to 0.95 mm sq, or about 5.0 mm sq to 0.80 mm sq, or about 5.0 mm sq to 6.5 mm sq, or about 6.5 mm sq to 1.1 mm sq, or about 6.5 mm sq to 0.95 mm sq, or about 6.5 mm sq to 0.80 mm sq, or about 0.80 mm sq to 1.1 mm sq, or about 0.80 mm sq to 0.95 mm sq, or about 0.95 mm sq to 1.1 mm sq. In other embodiments, the light source (140) is the laser fiber itself which serves to illuminate the field and to provide energy to the insertion tip, which, when energized, provides or reflects light to the field.

In some embodiments, the image sensor (130) comprises a complementary metal-oxide-semiconductor (CMOS) sensor. In other embodiments, the image sensor (130) comprises a charge-coupled device (CCD).

In certain embodiments, the image sensor (130) is 1.1 mm square (sq). In other embodiments, the image sensor (130) is about 0.2 mm sq to 1.6 mm sq, or about 0.2 mm sq to 1.4 mm sq, or about 0.2 mm sq to 1.2 mm sq, or about 0.2 mm sq to 1.0 mm sq, or about 0.2 mm sq to 1.1 mm sq, or about 0.2 mm sq to 0.8 mm sq, or about 0.2 mm sq to 0.6 mm sq, 0.2 mm sq to 0.4 mm sq, or about 0.4 mm sq to 1.6 mm sq, or about 0.4 mm sq to 1.4 mm sq, or about 0.4 mm sq to 1.2 mm sq, or about 0.4 mm sq to 1.0 mm sq, or about 0.4 mm sq to 1.1 mm sq, or about 0.4 mm sq to 0.8 mm sq, or about 0.4 mm sq to 0.6 mm sq, or about 0.6 mm sq to 1.6 mm sq, or about 0.6 mm sq to 1.4 mm sq, or about 0.6 mm sq to 1.2 mm sq, or about 0.6 mm sq to 1.1 mm sq, or about 0.6 mm sq to 1.0 mm sq, or about 0.6 mm sq to 0.8 mm sq, or about 0.8 mm sq to 1.6 mm sq, or about 0.8 mm sq to 1.4 mm sq, or about 0.8 mm sq to 1.2 mm sq, or about 0.8 mm sq to 1.1 mm sq, or about 0.8 mm sq to 1.0 mm sq, or about 1.0 mm sq to 1.6 mm sq, or about 1.0 mm sq to 1.4 mm sq, or about 1.0 mm sq to 1.2 mm sq, or about 1.0 mm sq to 1.1 mm sq, or about 1.1 mm sq to 1.6 mm sq, or about 1.1 mm sq to 1.4 mm sq, or about 1.1 mm sq to 1.2 mm sq, or about 1.2 mm sq to 1.6 mm sq, or about 1.2 mm sq to 1.4 mm sq, or about 1.4 mm sq to 1.6 mm sq, or about 1.6 mm sq to 1.8 mm sq.

In some embodiments, the image sensor (130) and the light source (140) are separate components. In some embodiments, the image sensor (130) and the light source (140) are combined together in a singular component. In some embodiments, the image sensor (130) and the light source (140) comprise a combined LED CMOS sensor.

In some embodiments, the device (100) is an endoscope. In some embodiments, the device (100) is a ureteroscope. In other embodiments, the device (100) is an angioscope, a biliary endoscope, or a laryngoscope. In further embodiments, the device (100) is a smaller endoscope for use in salivary ducts or a spinal canal.

In some embodiments, the endoscope devices (100) described herein are disposable. In other embodiments, the endoscope devices (100) described herein are reusable. In some embodiments, the endoscope devices (100) described herein are sterilizable. Without wishing to limit the present invention to any theory or mechanism, it is believed that a single use disposable device decreases and/or eliminates the risk of device contamination or breakage during reprocessing and/or sterilization. Thus, in accordance with an embodiment herein, the invention provides a novel endoscope (e.g., ureteroscope) that is disposable and single use, thereby eliminating the aforedescribed problems and the costs due to reprocessing.

The devices described herein may be passed up the ureter once, following which the stone undergoes laser lithotripsy, and all fragments/dust are aspirated under direct vision from the renal pelvis or ureter, thus leaving the kidney or ureter stone free. This eliminates the need for using a ureteral access sheath or stone basket. Also, when using the force sensor to pass the endoscope, it proceeds up the ureter at only 3 N; then, one might eliminate the placement of a ureteral stent at the end of the procedure.

In some embodiments, the force sensor may comprise an electromechanical force sensor. In some embodiments, the force sensor may comprise a mechanical force sensor. In other embodiments, the force sensor may comprise a handheld load-sensing device. In some embodiments, the force sensor may be adapted to reversibly attach to a device (100; e.g., at a proximal end). In some embodiments, the force sensor detects the amount of force applied to the proximal end of the device (100) during deployment of the device over the guidewire in a patient and outputs a force value representative thereof through an input/output interface. Other embodiments of force sensors that may be used in accordance with the present invention are disclosed in U.S. patent application Ser. No. 17/570,776, which has been herein incorporated by reference.

In some embodiments, the endoscope devices (100) described herein are wireless. In other embodiments, the endoscope devices (100) described herein are self-contained. In some embodiments, the devices (100) described herein are battery operated. In some embodiments, the image sensor (130) is wireless. In further embodiments, the devices described herein comprise a self-contained battery pack and are Bluetooth™ and/or WiFi enabled, thereby eliminating all electrical cords and cables.

The present invention may feature a method of removing a biopsied tissue, stone particle or fragment thereof from an individual. In some embodiments, the method comprises providing an endoscope device (100) comprising an insertion tip (165) at a distal end (101) of the insertion tube (160), an aspiration channel (110) disposed in the insertion tube (160), and having an outlet (115) at the insertion tip (165), at least one blocking bar (120) disposed at the outlet (115) of the aspiration channel (110), a plurality of working channels (150) disposed in the insertion tube (160) each having an outlet (155) at the insertion tip (165), an image sensor (130) and a light source (140) disposed at the insertion tip (165). In some embodiments, the at least one blocking bar (120) splits the outlet (115) of the aspiration channel (110) into multiple openings. The method further comprises removing the biopsied tissue or stone particle or fragment thereof from the individual via suction provided by the aspiration channel (110). In some embodiments, the biopsied tissue, stone particle or fragment thereof is removed via suction provided by the aspiration channel.

In some embodiments, the endoscope device (100) is an angioscope or a biliary endoscope.

In some embodiments, the biopsied tissue, stone particle, or fragment thereof is removed as part of a bronchoscopy, gastroscopy, duodenoscopy, small bowel endoscopy, colonoscopy, arthroscopy, laryngoscopy procedure and/or laparoscopic/robotic surgery. In other embodiments, the stone particle or fragment thereof is removed as part of a ureteroscopic and/or percutaneous nephrolithotomy procedure.

In other embodiments, the present invention features a method of performing a biopsy in a subject. In some embodiments, the method comprises providing an endoscope device (100) comprising an insertion tip (165) at a distal end (101) of the insertion tube (160), an aspiration channel (110) disposed in the insertion tube (160), and having an outlet (115) at the insertion tip (165), at least one blocking bar (120) disposed at the outlet (115) of the aspiration channel (110), a plurality of working channels (150) disposed in the insertion tube (160) each having an outlet (155) at the insertion tip (165), an image sensor (130) and a light source (140) disposed at the insertion tip (165). In some embodiments, the at least one blocking bar (120) splits the outlet (115) of the aspiration channel (110) into multiple openings. The further method comprises removing a biopsy sample from the subject via suction provided by the aspiration channel (110). In some embodiments, the biopsy sample is removed via suction provided by the aspiration channel (110). In some embodiments, the sample is a tumor and/or polyp.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A endoscope device (100) comprising:
a) an insertion tube (160) comprising an insertion tip (165) at a distal end (101) of the insertion tube (160);
b) an aspiration channel (110) disposed in the insertion tube (160), the aspiration channel (110) having an outlet (115) at the insertion tip (165);
c) at least one blocking bar (120) disposed at the outlet (115) of the aspiration channel (110), wherein the at least one blocking bar (120) splits the outlet (115) of the aspiration channel (110) into multiple openings, and
d) a plurality of working channels (150) disposed in the insertion tube (160), each having an outlet (155) at the insertion tip (165); and
e) an image sensor (130) and a light source (140) disposed at the insertion tip (165).

2. The endoscope device (100) of claim 1, wherein the outlet (115) of the aspiration channel (110) occupies at least half of a space on the insertion tip (165), wherein the outlets (155) of the working channels (150), image sensor (130), and light source (140) occupies the remaining space on the insertion tip (165).

3. The endoscope device (100) of claim 1, wherein the outlet (115) of the aspiration channel (110) is semicircular in shape.

4. The endoscope device (100) of claim 1, wherein the at least one blocking bar (120) splits the outlet (115) of the aspiration channel (110) equally.

5. The endoscope device (100) of claim 4, wherein the at least one blocking bar (120) is centrally disposed at the outlet (115) of the aspiration channel (110) at a 90° angle.

6. The endoscope device (100) of claim 1, wherein the at least one blocking bar (120) splits the outlet (115) of the aspiration channel (110) unequally.

7. The endoscope device (100) of claim 6, wherein the at least one blocking bar (120) disposed at the outlet (115) of the aspiration channel (110) at a 60° or 30° angle.

8. The endoscope device (100) of claim 1, wherein the insertion tube (160) further comprises a divider (170) disposed longitudinally within the insertion tube (160) such that an interior space of the insertion tube (160) is divided into two lumens, wherein one of the lumens forms the aspiration channel (110) and the other lumen contains the plurality of working channels (150), the image sensor (130), and the light source (140).

9. The endoscope device (100) of claim 1, wherein the plurality of working channels (150) comprises an irrigation port, a basket port, a laser port, or a combination thereof.

10. The endoscope device (100) of claim 1, wherein the endoscope device (100) is rigid or flexible.

11. The endoscope device (100) of claim 1, wherein the image sensor (130) comprises a complementary metal-oxide-semiconductor (CMOS) sensor or a charge-coupled device (CCD)

12. The endoscope device (100) of claim 1, wherein the light source (140) comprises an LED (light-emitting diode) or a fiber optic.

13. The endoscope device (100) of claim 1, wherein the image sensor (130) and the light source (140) are combined and comprise a combined light-emitting diode (LED) complementary metal-oxide-semiconductor (CMOS) sensor.

14. A method of removing a biopsied tissue, stone particle or fragment thereof from an individual, the method comprising:
a) providing a device (100) comprising
i. an insertion tube (160) comprising an insertion tip (165) at a distal end (101) of the insertion tube (160);
ii. an aspiration channel (110) disposed in the insertion tube (160), the aspiration channel (110) having an outlet (115) at the insertion tip (165);
iii. at least one blocking bar (120) disposed at the outlet (115) of the aspiration channel (110), wherein the at least one blocking bar (120) splits the outlet (115) of the aspiration channel (110) into multiple openings, and
iv. a plurality of working channels (150) disposed in the insertion tube (160), each having an outlet (155) at the insertion tip (165); and
v. an image sensor (130) and a light source (140) disposed at the insertion tip (165); and
b) removing the biopsied tissue, stone particle or fragment thereof from the individual via suction provided by the aspiration channel (110).

15. The method of claim 14, wherein the device is an angioscope, or a biliary endoscope.

16. The method of claim 14, wherein the biopsied tissue, stone particle or fragment thereof is removed as part of a bronchoscopy, gastroscopy, duodenoscopy, small bowel endoscopy, colonoscopy, arthroscopy, laryngoscopy procedure and/or laparoscopic/robotic surgery.

17. The method of claim 14, wherein the stone particle or fragment thereof is removed as part of a ureteroscopic or percutaneous nephrolithotomy procedure.

18. A method of performing a biopsy in a subject, comprising:
a) providing a device (100) comprising
i. an insertion tube (160) comprising an insertion tip (165) ata distal end (101) of the insertion tube (160);
ii. an aspiration channel (110) disposed in the insertion tube (160), the aspiration channel (110) having an outlet (115) at the insertion tip (165);
iii. at least one blocking bar (120) disposed at the outlet (115) of the aspiration channel (110), wherein the at least one blocking bar (120) splits the outlet (115) of the aspiration channel (110) into multiple openings, and
iv. a plurality of working channels (150) disposed in the insertion tube (160), each having an outlet (155) at the insertion tip (165); and
v. an image sensor (130) and a light source (140) disposed at the insertion tip (165); and
b) removing a biopsy sample from the subject via suction provided by the aspiration channel (110).

19. The method of claim 18, wherein the sample is a tumor or polyp.
